(19) United States
(12) Patent Application Publication
(10) Pub. No.: US 2017/0202928 A1
(43) Pub. Date: Jul. 20, 2017

(54) METHOD OF FEEDING ANIMALS PHYTASE PRODUCTS

(71) Applicant: PURINA ANIMAL NUTRITION LLC, Shoreview, MN (US)

(72) Inventors: Bill L. Miller, Labadie, MO (US); James D. Caldwell, St. Clair, MO (US)

(21) Appl. No.: 14/995,829

(22) Filed: Jan. 14, 2016

(51) Int. Cl. A61K 38/46; A23J 1/00; A23C 11/06; A23C 9/152; A61K 38/16; A23K 50/60

(52) U.S. Cl. CPC A61K 38/465; A61K 38/168; A23K 50/60; A23C 11/06; A23C 9/152; A23J 1/006

(57) ABSTRACT

Methods involve feeding livestock animals a milk replacer, where the milk replacer includes non-milk proteins and phytase. Prior to feeding the milk replacer, the phytase may be activated through heat and moisture treatment. The activated phytase may rest for an activation period and the milk replacer containing the activated phytase may be fed to the animal thereafter. The livestock animals may be calves, and may be between about 0 and about 3 weeks of age. In response to ingesting the milk replacer, the calves may increase a rate of weight gain.

### TECHNICAL FIELD

Implementations relate to livestock animal feed products and methods of feeding such products to animals. More particularly, implementations provide methods of feeding milk replacers comprised of non-milk proteins supplemented with phytase to livestock animals resulting in improved performance.

### BACKGROUND

Young animals require adequate nutrition for healthy growth and development. Robust growth is especially important for livestock animals, which are a commodity raised to produce food products including milk and meat. The time it takes livestock to mature, particularly to gain weight, is important when assessing whether the animal is ready to produce milk or is ready to go to market. A number of feeding systems have been used to enhance weight gain of livestock beginning at a young age and may include feeding techniques prior to and after weaning. These techniques may involve providing milk replacer products to the animals.

Milk ingredient-based products designed to replace mother's milk are common. These products are frequently used to permit the use of mother's milk for more cost-effective applications and can also be employed in emergency situations where mother's milk is not available. Non-milk proteins derived from oilseeds or cereal protein products have been used for decades in these milk replacer formats due to potential allergy concerns or for cost-savings reasons. Today in animal nutrition, these non-milk proteins (NMPs) are increasingly added due to the escalating cost of milk protein products. However, this substitution of NMPs in formulations for young animals is often problematic. Neonate growth and other performance criteria resulting from feeding milk replacers containing NMPs may be reduced compared to that of animals fed only milk proteins. An increased incidence of diarrhea may also be observed when diets containing NMPs are fed. Given the already widespread use of NMPs in milk replacers, these health and performance issues could worsen without improved methods of feeding young livestock animals.

### SUMMARY

Implementations provide methods of feeding a milk replacer to livestock animals that includes non-milk proteins and phytase.

In various implementations and alternatives, the livestock animals may ingest about 0.01 to about 8 grams of phytase per head per day.

In various implementations and alternatives, the non-milk proteins may include vegetable proteins.

In additional implementations and alternatives, the livestock animals may be calves. In such cases, the calves may ingest about 0.5 to about 3 pounds of milk replacer per head per day.

In addition or alternatively, the milk replacer may include about 18 to about 30 wt % protein by dry weight, and/or about 15 to about 31 wt % fat by dry weight.

In various implementations and alternatives, the livestock animals may be fed the milk replacer between about birth and about 3 weeks of age, between about birth and about completion of the weaning process, or between about birth until weaning.

In various implementations and alternatives, the livestock animals may increase feed efficiency, decrease a rate of milk replacer refusal, and/or increase a rate of weight gain in response to ingesting the milk replacer.

In addition or alternatively, the phytase may be derived from a species of bacteria, which may be selected from the group of genera including: Escherichia, Buttiauxella, Aspergillus, or Thermomyces. In such cases, the bacteria may be genetically modified.

In addition or alternatively, the feeding method may further involve activating the phytase prior to feeding the animal, which may involve combining the phytase with an aqueous material having a temperature of about 90° F. to about 160° F. In such implementations, the activated phytase may rest for an activation period of about 10 to about 90 minutes prior to feeding the animal.

In additional implementations and alternatives, the livestock animals may also be fed a starter feed. In such cases, the livestock animals may increase consumption of the starter feed in response to ingesting the milk replacer.

In another implementation, a method of feeding milk replacer to calves is provided, where the milk replacer includes non-milk proteins and phytase, and the calves are between about 0 and about 3 weeks of age. In response to ingesting the milk replacer that includes non-milk proteins and phytase, the calves increase a rate of weight gain.

In such cases, the calves may ingest between about 0.01 and about 8 grams of phytase per head per day, and/or the non-milk proteins may include vegetable protein.

### DETAILED DESCRIPTION

Enabling young livestock animals to maximize the nutritional benefit derived from their feed is critical for both short-term and long-term growth. The poor performance and health issues associated with diets containing NMPs generally occur when these diets are fed within the first few weeks after birth, at a time when the effects of insufficient nutrition can be exacerbated by vulnerable, undeveloped gastrointestinal systems. The digestive system of calves, for example, has particular difficulty with plant proteins during the first two to three weeks of life. Because milk replacers may serve as the primary source of nutrients for livestock animals shortly after birth, methods designed to enhance digestion of the NMPs commonly present in milk replacer formulas offer particular advantages. Applicant's knowledge regarding NMPs and their physical interactions with other milk replacer components guided the experimentation that ultimately led to the methods disclosed herein. Specifically, one of the nutrients supplied by plants, phosphorus, associates with NMPs in a way that diminishes nutrient digestibility.

In cereal grains and legume plants, phytic acid is a major storage form of phosphorus. These types of plants contain up to 5 percent (weight basis) of phytic acid. Phytic acid forms an array of complexes with proteins and other minerals—termed “phytate.” The presence of complexed phytic acid/mineral/proteins reduces the solubility and digestibility of nutrients, and can negatively impact livestock growth. To combat the productive losses due to phytate, exogenous phytase may be used. Phytase is a phosphatase enzyme of bacterial origin that catalyzes the removal of phosphate groups from their substrates, and its inclusion in dry feed facilitates the cleaving of phosphorus, proteins and other minerals from the phytate complex.

By experimentally manipulating the liquid diets of young livestock animals using phytase additives, Applicant discovered that neonate livestock animals meaningfully respond to enhanced levels of phytase when fed liquid diets that contain NMPs. The significant result of feeding NMPs and phytase in milk replacer is a surprising improvement over prior phytase uses. In particular, the milk replacer containing phytase improved animal performance even though excess phosphorus levels were provided, indicating that the liberation of phosphate groups may not be the only beneficial effect of phytase when used in milk replacers with NMPs. It is believed that increased protein solubility may be particularly important to the young animal, especially during this critical early period of the young animal's life when sequestration of valuable proteins within phytate moieties would otherwise shield them from proper digestion. Because the phytase enzyme is presented to the animal in an aqueous format, the activity of enzymes may be optimized. In particular, hydrating the milk replacer powder at elevated temperatures just prior to feeding the liquid milk replacer may provide sufficient heat and moisture for phytase activation, diminishing the enzyme's reliance on suboptimal moisture levels in the animal's gastrointestinal tract for activation. Accordingly, methods of feeding milk replacer products comprised of phytase are provided herein.

### Milk Replacer Compositions Containing Phytase

Milk replacers of the present disclosure may include or be admixed with phytase.

Sources of phytase may include, but are not limited to: microbial species that may belong to the Escherichia, Buttiauxella, Aspergillus, and/or Thermomyces genus. Phytase may be derived from a single source or multiple sources of bacteria. In some embodiments, the phytase may be derived from a genetically modified strain of Aspergillus oyzae. Phytase may also be derived from plant-based or fungal sources. The bacterial and/or plant sources of phytase may be transgenic.

The phytase used according to the methods disclosed herein exhibit phosphatase activity, allowing it to enzymatically liberate at least one phosphate group from a phytate complex. The chemical name for one exemplary form of phytase is myo-inositol hexakisphosphate phosphohydrolase.

The phytase content of the phytase composition added to a milk replacer according to the methods herein may vary depending on the processes used to manufacture the phytase composition, the sources of phytase used, and the desired phytase activity levels. Because no uniform phytase unit system currently exists, various feed manufacturers have developed phytase unit equivalencies for different phytase compositions. Phytase levels may be conveyed as concentrations. In some embodiments, the phytase content may range from about 100,000 to about 5,000,000 phytase units, about 1,000,000 to about 4,000,000 phytase units, about 1,500,000 to about 3,500,000 phytase units, about 2,000,000 to about 3,000,000 phytase units, about 2,400,000 to about 2,800,000 phytase units, or about 2,600,000 to about 2,700,000 phytase units per pound of the phytase composition.

The phytase content of the milk replacer may vary and may be adjusted according to the needs and/or condition of the animal. Raising phytase concentrations may be achieved by adding fixed amounts of increasingly-concentrated phytase compositions to an amount of milk replacer. Alternatively, phytase concentration may be increased by adding a greater amount of a phytase composition to a fixed amount of milk replacer. In some embodiments, about 0.1 to about 10 pounds, about 0.5 to about 7.5 pounds, about 1 to about 5 pounds, about 1 to about 3 pounds, about 0.5, about 1, about 2, about 3, about 4, about 5, about 6, about 7, about 8, about 9, or about 10 pounds of phytase composition may be added per ton (i.e., 2000 lbs.) of dry milk replacer powder. The final phytase content of the composition administered to animals according to the methods disclosed herein may be about 200 to about 10,000 phytase units, about 200 to about 5,000 phytase units, about 200 to about 2,500 phytase units, about 200 to about 600 phytase units, about 1,000 to about 1,200 phytase units, about 1,700 to about 1,900 phytase units, about 1,000 to about 2,500, about 1,000 to about 2,300, or about 2,200 to about 2,300 phytase units per pound of dry milk replacer powder. For example, about 1 pound of concentrated Ronozyme HiPhos 5000 (DSM) phytase may be included in 1 ton of dry milk replacer to produce a composition with 1,140 phytase units per pound of dry milk replacer.

Milk replacers of the present disclosure may be produced according to traditional methods in which the fat and protein components of milk replacers are spray dried and combined into a milk replacer powder comprised of soluble or at least suspendable ingredients. Spray drying processes generally involve maintaining a spray dryer at temperatures between 100° C. to 200° C. so that the spray dried component rapidly heats and loses moisture. Following spray drying, the spray dried powder is subjected to a subsequent heating step, such as in a dryer drum, with an air temperature of between 100° C. to 200° C. in order to further reduce the moisture content of the powder.

The nutrient profile of the milk replacer generally includes fat and protein. The fat content may range from about 2.25 to about 4.7 wt % of the hydrated milk replacer or from about 15 to about 31 wt % of the milk replacer powder. The level of fat may be tailored for a target animal, and for instance, calf milk replacers may have the aforementioned fat content of between about 15 and about 31 wt % of the powder. In a more particular example, traditional calf milk replacers may include fat from about 20 to about 25 wt % of the powder or about 3 to about 3.75 wt % of the hydrated milk replacer, and full potential calf milk replacers may include fat from about 25 to about 31 wt % of the powder or about 3.75 to about 4.7 wt % of the hydrated milk replacer.

Predominant fat sources may be lard, tallow, palm kernel, or coconut oils, alone or in combination. In addition, some fat from lecithin and residual fat (e.g., butter fat, milkfat, or both) may contribute to the fat content in milk replacers.

Protein in milk replacers typically ranges from about 2.2 to about 5.1 wt % of the hydrated milk replacer or about 18 to about 30 wt % of the powder. For traditional calf milk replacers, the protein content may be about 22 wt % of the powder or about 3.3 wt % of the rehydrated milk replacer, and milk replacers formulated for enhanced performance, such as full potential milk replacers, may include protein at about 26 to about 28% of the powder or about 3.9 wt % to about 4.8 wt % of the rehydrated milk replacer.

Protein may be sourced from animal (e.g., milk, plasma, egg, and red blood cells) and vegetable sources and combinations thereof. Milk-derived protein sources are generally referred to as milk proteins and may include whey, casein, skim milk, sodium caseinate, and calcium caseinate. Non-milk proteins (NMPs), such as vegetable protein (e.g., soy protein, hydrolyzed soy protein, hydrolyzed soy protein modified, soy protein isolate, wheat concentrates, wheat isolates, pea concentrates, pea isolates, and/or potato proteins), animal protein (e.g., plasma such as bovine or porcine plasma, egg and red blood cells), and single cell protein, alone or in combination, may be included as a protein source in the milk replacer. Non-milk proteins may contain varying levels of phosphorous. For instance, phosphorous may be present at about 0.65 wt % of soybean meal, at about 0.78 wt % of soy protein isolate, at about 0.68 wt % of hydrolyzed soy protein modified, at about 1.0 wt % of dehulled canola meal, and each of these components may be present in NMP-containing milk replacers. NMPs may account for up to from 1 to about 65%, from about 50 to about 65%, about 55 to 65%, about 55 to 60%, or up to or at about 60 or about 65% of the total protein content, with the balance of protein derived from milk protein; while milk protein may account for about 35 to 99%, about 35 to about 50%, about 35 to 45, about 40 to 45, up to about 40%, or up to about 35% of the total protein content in the milk replacer.

Because the NMPs contain phosphorous and NMPs are generally a component of the milk replacers of the present disclosure, phytate present in such milk replacers can reduce the availability of phosphorous, calcium, protein, and other nutrients to the animal. Accordingly, the milk replacers used according to the methods herein may include adequate levels of total calcium and phosphorus, which comprises levels of phosphorus sufficient to satisfy the nutritional requirements of livestock animals even without the phosphorus-cleaving activity provided by exogenous phytase. In some embodiments, the levels of phosphorus and/or calcium may even be in excess of the animals' nutritional requirements. In other embodiments, the calcium and/or phosphorus content may vary according to the specific needs of the animal. In some embodiments, calcium content may be about 0.5 to about 1.5 wt %, about 0.70 to about 1.2 wt %, about 0.80 to about 1.0 wt %, or about 0.9 wt % of the powdered milk replacer. In additional or alternative embodiments, phosphorus content may be about 0.3 to about 1.2 wt %, about 0.5 to about 1 wt %, about 0.6 to about 0.8 wt %, or about 0.7 wt % of powdered milk replacer.

### Methods of Feeding Milk Replacers Containing Phytase

Methods of feeding animals may involve feeding the animals milk replacer containing phytase. This approach may include obtaining a phytase additive and combining it with a milk replacer just prior to feeding. Alternatively, the milk replacer may contain the phytase.

Generally, animals are offered a fixed amount of milk replacer per day, which may form all or a portion of the young animal's daily feed ration. Prior to the onset of weaning, the milk replacer in the feed ration may be offered twice per day, and may generally be divided into equal parts.

Milk replacers may be fed in traditional settings at a rate of about 1.25 pounds per head per day on a dry weight basis during the first week of life. Thereafter, the animal may be offered about 1.5 pounds of milk replacer per head per day on a dry weight basis. At the onset of weaning, the animal may be offered one feeding per day, totaling about 0.75 pounds of milk replacer per head per day on a dry weight basis.

In enhanced feed settings, full potential milk replacers may be fed at a rate of at least about 1.6 pounds up to about 3.0 pounds per head per day on a dry weight basis. For instance, in the first week of life, young animals, such as calves, in a full potential setting may be offered about 1.6 pounds or more (e.g., up to about 1.9 pounds) of milk replacer per head per day on a dry weight basis. From the second week of life onward, such animals in a full potential setting may be offered the same amount (about 1.6 pounds) of milk replacer or may be offered up to 3.0 pounds of milk replacer per head per day on a dry weight basis. Thereafter, the amount of milk replacer offered to the young animal may be maintained or the level may decrease, for example, depending on the timing of the onset of weaning.

Prior to feeding the animals, the milk replacers are hydrated with water or another aqueous material. According to certain implementations, the phytase may be activated prior to feeding the phytase-containing milk replacer to the animal. For example, the aqueous material may be combined with the milk replacer containing phytase when the aqueous material is at elevated temperatures, such as at a temperature of about 90° F. to about 160° F., about 90° F. to about 130° F., about 100° F. to about 130° F., about 100° F. to about 120° F., about 90° F. to about 110° F., about 100° F. to about 110° F., at least 100° F., at about or up to about 110° F., or up to about 160° F. In another example, an amount of a phytase additive may be admixed with the rehydrated milk replacer. Here, the rehydrated milk replacer may be at any of the aforementioned elevated temperatures when the phytase is added. Alternatively, the phytase may be activated just prior to combining with the milk replacer. In this example, the phytase may be mixed with the aqueous material at any of the aforementioned elevated temperatures, and then the activated phytase may be admixed with the milk replacers in connection with rehydration of the milk replacers. The phytase may be in solid or liquid form prior to activation, and its form may be dependent on whether the phytase is present in a calf milk replacer product, in which case it may be preferable to have the phytase in solid form, or whether it is admixed with the calf milk replacer, in which case the phytase may be in solid or liquid form.

In implementations where the phytase is heat and moisture activated, for instance, according to the preceding examples, the activated enzyme may rest for a period about 10 to about 90 minutes, about 10 to 60 minutes, about 30 to 90 minutes, about 30 to 60 minutes, or about 60 to 90 minutes, prior to feeding the milk replacer to the animal. This activation period may enhance the ability for the activated enzyme to function upon ingestion by the animal. Both the moisture content and the temperature of the enzyme in the aqueous solution may be higher than the moisture and temperature of the animal, and consequently, the enzyme may more rapidly activate under these conditions as compared to the animal ingesting the phytase in the inactive state, whether it be in an aqueous mixture or in solid feedstuffs (e.g., dry feed), which results in delayed activation and activation at a slower rate due to the lower moisture content in the animal digesta and/or the lower basal temperature of the animal.

The amount of phytase fed to and available for consumption by the livestock animals may be based on the animal's size, health status, age, and activity level. For example, the amount of phytase may be increased if the animal exhibits symptoms consistent with gastrointestinal disease. Phytase levels may also be increased for animals consuming a greater level of NMPs, such as plant proteins. Alternatively, phytase levels may be held constant or decreased as livestock animals' digestive systems mature and become better equipped to digest plant proteins. Phytase levels may also be adjusted according to phytate levels present in an animal's diet. Various feed additives or enzymes fed concurrently with the milk replacer comprising phytase may also impact the level of phytase used. In some embodiments, a livestock animal may be fed about 0.01 to about 8 grams, about 0.01 to about 6 grams, about 0.1 to about 4 grams, or about 0.1 to about 3 grams of phytase per day.

The methods disclosed herein confer significant benefit on young livestock animals in digesting NMPs. In a preferred embodiment, livestock animals are fed between birth and about 3 weeks of age. In other embodiments, the livestock animals are fed between birth and about 2, 4, or 5 weeks of age. In alternative embodiments, the livestock animals are fed between birth and about 6 or 7 weeks of age. In still other embodiments, the livestock animals are fed between birth and completion of the weaning process. Young livestock animals may be fed according to the methods disclosed herein over the entirety of any of the aforementioned periods, or for shorter subsets of time falling within or overlapping with these periods. Where the young livestock animals are fed phytase through weaning, the phytase level ingested may be reduced by about half compared to phytase levels ingested pre-weaning. For instance, where the animal ingests 3 grams of phytase per day pre-weaning, the weaning animal may ingest 1.5 grams of phytase per day.

Livestock animals at particular weight ranges may especially benefit from the feeding methods disclosed herein. For example, livestock animals weighing from about 75 to about 150 pounds, about 85 to about 140 pounds, or about 90 to 135 pounds may be particularly responsive to the feeding methods disclosed herein. In some implementations, the age of such young livestock animals at these weights may correspond to 2, 3, 4, 5, 6, or 7 weeks of age, or the age of the completion of the weaning process. In further implementations, young livestock animals fed the milk replacers of the present disclosure, upon reaching between 135 and 150 pounds may be fed milk replacers containing no, or reduced levels of phytase. In such implementations, the age of the young livestock animal may correspond to 2, 3, 4, 5, 6, or 7 weeks of age, or the age of the completion of the weaning process.

In addition to milk replacer, starter feed may be offered to the young animals on an ad libitum basis. Starter feeds, such as calf starter feeds, may include a mixture of one or more of corn, soybean meal, wheat middlings, oats, molasses, fat, ground cotton seed hulls, distillers grains, calcium carbonate, salt, and macronutrients and micronutrients. The starter feed may contain about 45 to 50% coarse ingredients such as corn, soy, and oats; about 16 to 22% protein; about 2 to 3% fat; about 5 to 6% fiber (determined on a NIR basis); about 7% acid detergent fiber; about 6% molasses; and the balance including a mixture of other nutrients. The amount of starter feed offered to the young animals may increase as the animals progress through the weaning process.

In addition to milk replacer, forage may be provided to the young animals to promote optimal digestive health. Sources of forage may include grasses, long-stem hay, hay cubes, and hay pellets. The amount of forage offered or provided to the young animals may increase as the animals progress through weaning.

Ingestion of milk replacers containing phytase by livestock animals according to the present disclosure has been found to improve performance. For example, young livestock animals may increase weight gain, starter feed intake, and/or milk replacer intake. In addition or alternatively, young livestock animals may improve feed-to-gain ratio such that less feed consumption is required per unit of weight gain. Each of these effects may occur between about 0 and about 4 weeks of age, about 0 and about 5 weeks of age, or about 0 and about 6 weeks of age. For instance, young livestock animals may increase weight gain between birth and about 4 weeks of age by a total of at least about 4 pounds. In addition or alternatively, young livestock animals may increase body weight by at least about 30% between birth and about 4 weeks of age. In addition or alternatively, young livestock animals may increase starter feed intake between birth and about 4 weeks of age by a total of at least about 2 pounds. Additional or alternative embodiments may comprise an improvement in feed-to-gain ratio of at least about 10% between birth and about 4 weeks of age. Improved performance may also result in a reduction of the age of freshening or the onset of lactation, thereby reducing the cost of milk production; or may cause the animal to increase weight gain from early age, which may reduce the cost of beef production. Enhanced performance may be the result of an increase in non-milk protein solubility caused by phytase activity.

Performance improvements in weight gain, milk replacer intake, dry feed intake, and/or feed-to-gain ratio may be significant for animals fed milk replacer containing enhanced levels of phytase (e.g., about 1000 to about 2,500 PU per pound of milk replacer). These performance improvements may occur between birth and about 1, 2, 3, 4, 5, 6, or 7 weeks of age and may be realized in comparison to animals fed milk replacer diets without phytase and/or animals fed milk replacer containing lower phytase levels (e.g., about 570 phytase units per pound of milk replacer). For animals fed lower phytase levels, performance improvements may still occur, but these improvements may be more modest and/or may occur at a slower rate compared to animals fed enhanced phytase levels. The number, size, and/or rate of performance improvements may increase as phytase levels increase.

Implementations of the present disclosure are more particularly described in the following calf trials that are for illustrative purposes only. Numerous modifications and variations are within the scope of the present disclosure as will be apparent to those skilled in the art.

### Examples

Calf Trial 1

This study was conducted to assess the ability of calf milk replacer supplemented with phytase to increase calf performance. Measured performance parameters included weight gain, milk replacer consumption, and starter feed consumption. By measuring the effects of phytase at a range of concentrations, this study revealed the significant improvement in calf performance that may be achieved by feeding calves according to the methods disclosed herein.

One-hundred twenty-eight bull calves, each between 2 and 5 days old, were sourced from Wisconsin, co-mingled, and shipped to Eastern Missouri. Upon arrival, all calves were treated per standard receiving protocol. The following day, calves were stratified by initial body weight and immunoglobulin status, assigned to experimental treatments, and the tests were initiated.

Twenty-one calves were allotted to each of 5 treatments where each was offered 1.50 lbs. per day of the same conventional milk replacer (Land O' Lakes Maxi-Care Plus), which contains 25% crude protein and 17% fat. The milk replacer powder was fed daily in two meals (0.75 lbs./feeding) after reconstituting with 110° F. water. The milk replacer powder was fed as an 11.6% solids solution. The protein in the milk replacer contained about 58% NMPs, which comprised hydrolyzed soy protein modified, soy protein isolate, and bovine plasma. The balance of protein was derived from milk protein, i.e., about 42% of the protein. Calves received the same 20% crude protein starter (Purina AmpliCalf 20) throughout the trial ad libitum. The treatments provided to the test animals comprised different concentrations of phytase, expressed in phytase units per pound (PU/lb.). Treatments for the four-week trial included: negative control with no phytase; control plus 570 PU/lb. Ronozyme phytase; control plus 1140 PU/lb. Ronozyme phytase; control plus 2280 PU/lb. Ronozyme phytase; and control plus 1140 PU/lb. Quantum Blue phytase. Ronozyme HiPhos was sourced from DSM Nutritional Products, Parsippany, N.J. and Quantum Blue 40P was sourced from AB Vista Feed Ingredients, Plantation, Fla. All milk replacer treatments were formulated to contain 0.90 wt % total calcium and 0.70 wt % total phosphorus, which was in excess of the animals' nutritional requirements.

TABLE 1
Performance by calves fed milk replacer with different
phytase levels or sources

| | | | | | 1140 PU | |
|---|---|---|---|---|---|---|
| | No | 570 PU | 1140 PU | 2280 PU | Quantum | |
| Item | Phytase | Ronozyme | Ronozyme | Ronozyme | Blue | SEM |
| Initial | 3.79 | 3.86 | 3.79 | 3.80 | 3.84 | 0.23 |
| Ig | | | | | | |
| Initial | 98.4 | 98.6 | 98.8 | 99.0 | 98.8 | 1.38 |
| weight, | | | | | | |
| lbs. | | | | | | |
| Final | 127.8 | 130.0 | 132.4 | 134.4 | 131.9 | 2.37 |
| weight, | | | | | | |
| lbs. | | | | | | |

Avg. Period Gain, lbs.

| | | | | | | |
|---|---|---|---|---|---|---|
| Week 1 | 7.56 | 8.44 | 8.74 | 8.10 | 7.66 | 0.71 |
| Week 2 | 2.87 | 2.79 | 4.33 | 4.86 | 4.66 | 0.91 |
| Week 3 | 9.01 | 8.84 | 9.49 | 9.81 | 9.44 | 0.95 |
| Week 4 | 9.99a | 11.30ab | 11.04ab | 12.66b | 11.34ab | 0.95 |
| Total | 29.44a | 31.37ab | 33.61ab | 35.42b | 33.09ab | 1.95 |
| gain | | | | | | |

Avg. Period Milk Replacer Intake, lbs. (DM Basis)

| | | | | | | |
|---|---|---|---|---|---|---|
| Week 1 | 10.07a | 10.29ab | 10.38b | 10.39b | 10.21ab | 0.08 |
| Week 2 | 10.42ab | 10.32a | 10.47b | 10.44b | 10.45b | 0.04 |
| Week 3 | 10.44 | 10.41 | 10.46 | 10.47 | 10.46 | 0.03 |
| Week 4 | 10.47 | 10.47 | 10.47 | 10.47 | 10.47 | 0.05 |
| Total | 41.40a | 41.50ab | 41.79b | 41.77b | 41.59ab | 0.11 |
| Milk | | | | | | |
| Replacer | | | | | | |
| Intake | | | | | | |

Period Dry Feed Intake, lbs. (DM Basis)

| | | | | | | |
|---|---|---|---|---|---|---|
| Week 1 | 0.19 | 0.20 | 0.25 | 0.31 | 0.34 | 0.07 |
| Week 2 | 1.13 | 1.18 | 1.38 | 1.90 | 1.53 | 0.36 |
| Week 3 | 3.37 | 3.62 | 3.87 | 4.98 | 3.94 | 0.67 |
| Week 4 | 6.28a | 7.51ab | 7.25ab | 8.96b | 7.61ab | 0.89 |
| Total | 10.98 | 12.51 | 12.75 | 16.15 | 13.42 | 1.88 |
| Dry | | | | | | |
| Feed | | | | | | |
| Intake | | | | | | |

Feed: Gain

| | | | | | | |
|---|---|---|---|---|---|---|
| Weeks | 1.90b | 1.82ab | 1.64a | 1.72ab | 1.71ab | 0.09 |
| 1-4 | | | | | | |

Means in the same row not followed by a common letter differ (P<0.05) using LSD procedure.

The data from Table 1 confirm that week 4 gains and total gains were numerically greater for animals fed phytase at all levels compared to the negative control animals fed no phytase; and gains were significantly improved for animals consuming the milk replacer containing 2280 PU/lb. Ronozyme phytase compared to the negative control animals fed no phytase. Average period milk replacer intakes from week 1 and total milk replacer intakes numerically increased for animals fed phytase at all levels compared to the negative control; and average period milk replacer intakes were significantly higher (P<0.05) for animals fed 1140 and 2280 PU/lb. Ronozyme phytase compared with the control. Average dry feed intake across weeks 1 through 4 and total dry feed intake was numerically greater for animals fed phytase at all levels compared to the negative control, and week 4 dry feed intake increased significantly for animals fed 2280 PU/lb. Ronozyme phytase compared with control animals (P<0.05). The feed-to-gain ratio improved for animals fed phytase at all levels compared with the negative control; and was significantly improved (P<0.05) in animals fed 1140 PU/lb. Ronozyme compared with the control.

In addition to the ANOVA assessment, the control and Ronozyme data from this calf trial were subjected to a linear contrast. This regression indicated a significant linear benefit to phytase inclusion for final weight (P=0.04), total gain (P=0.02), milk replacer intake (P=0.01), and feed efficiency (P=0.06), as displayed in Table 2.

TABLE 2
Performance by calves fed milk replacer with either no phytase or different levels of
phytase

| | No | 570 PU | 1140 PU | 2280 PU | | Linear |
|---|---|---|---|---|---|---|
| Item | Phytase | Ronozyme | Ronozyme | Ronozyme | SEM^{a} | P-value^{b} |
| Initial weight, lb. | 98.4 | 98.6 | 98.8 | 99.0 | 1.39 | 0.76 |
| Final weight, lb. | 127.8 | 130.0 | 132.4 | 134.4 | 2.30 | 0.04 |

Avg. Period Gain, lb.

| | | | | | | |
|---|---|---|---|---|---|---|
| Week 1 | 7.56 | 8.44 | 8.74 | 8.10 | 0.73 | 0.54 |
| Week 2 | 2.87 | 2.79 | 4.33 | 4.85 | 0.88 | 0.06 |
| Week 3 | 9.01 | 8.84 | 9.49 | 9.81 | 0.98 | 0.50 |
| Week 4 | 9.99 | 11.30 | 11.04 | 12.66 | 0.95 | 0.08 |
| Total gain | 29.44 | 31.37 | 33.61 | 35.42 | 1.92 | 0.02 |

Avg. Period Milk Replacer Intake, lb. (DM Basis)

| | | | | | | |
|---|---|---|---|---|---|---|
| Week 1 | 10.07 | 10.29 | 10.38 | 10.39 | 0.07 | <0.01 |
| Week 2 | 10.42 | 10.32 | 10.47 | 10.44 | 0.04 | 0.30 |
| Week 3 | 10.44 | 10.41 | 10.46 | 10.47 | 0.03 | 0.31 |
| Week 4 | 10.47 | 10.47 | 10.47 | 10.47 | 0.05 | 1.0 |
| Total Milk | 41.40 | 41.50 | 41.79 | 41.77 | 0.11 | 0.01 |
| Replacer Intake | | | | | | |

Period Dry Feed Intake, lb. (DM Basis)

| | | | | | | |
|---|---|---|---|---|---|---|
| Week 1 | 0.19 | 0.20 | 0.25 | 0.31 | 0.07 | 0.18 |
| Week 2 | 1.13 | 1.18 | 1.38 | 1.90 | 0.35 | 0.12 |
| Week 3 | 3.37 | 3.62 | 3.87 | 4.98 | 0.64 | 0.08 |
| Week 4 | 6.28 | 7.51 | 7.25 | 8.96 | 0.88 | 0.05 |
| Total Dry Feed Intake | 10.98 | 12.51 | 12.75 | 16.15 | 1.83 | 0.06 |

Feed: Gain

| | | | | | | |
|---|---|---|---|---|---|---|
| Weeks 1-4 | 1.90 | 1.82 | 1.64 | 1.72 | 0.10 | 0.10 |

^{a}SEM = Standard error of the mean.
^{b}P-value based on Linear contrast.

Finally, the control without phytase was contrasted to all treatments containing phytase and the results listed in Table 3. This analysis indicated that phytase feeding enhanced final weight (P=0.11), total gain (P=0.08), milk replacer intake (P=0.04), and feed efficiency (P=0.09).

TABLE 3
Performance by calves fed milk replacer with either no phytase or different
levels/sources of phytase

| | No | 570 PU | 1140 PU | 2280 PU | Quantum | | Contrast |
|---|---|---|---|---|---|---|---|
| Item | Phytase | Ronozyme | Ronozyme | Ronozyme | Blue | SEM^{a} | P-value^{b} |
| Initial weight, lb | 98.4 | 98.6 | 98.8 | 99.0 | 98.8 | 1.38 | 0.79 |
| Final weight, lb | 127.8 | 130.0 | 132.4 | 134.4 | 131.9 | 2.37 | 0.11 |

Avg. Period Gain, lb.

| | | | | | | | |
|---|---|---|---|---|---|---|---|
| Week 1 | 7.56 | 8.44 | 8.74 | 8.10 | 7.66 | 0.71 | 0.48 |
| Week 2 | 2.87 | 2.79 | 4.33 | 4.86 | 4.66 | 0.91 | 0.20 |
| Week 3 | 9.01 | 8.84 | 9.49 | 9.81 | 9.44 | 0.95 | 0.22 |
| Week 4 | 9.99 | 11.30 | 11.04 | 12.66 | 11.34 | 0.95 | 0.11 |
| Total gain | 29.44 | 31.37 | 33.61 | 35.42 | 33.09 | 1.95 | 0.08 |

Avg. Period Milk Replacer Intake, lb. (DM Basis)

| | | | | | | | |
|---|---|---|---|---|---|---|---|
| Week 1 | 10.07 | 10.29 | 10.38 | 10.39 | 10.21 | 0.08 | 0.01 |
| Week 2 | 10.42 | 10.32 | 10.47 | 10.44 | 10.45 | 0.04 | 0.93 |
| Week 3 | 10.44 | 10.41 | 10.46 | 10.47 | 10.46 | 0.03 | 0.74 |
| Week 4 | 10.47 | 10.47 | 10.47 | 10.47 | 10.47 | 0.05 | 1.0 |
| Total Milk | 41.40 | 41.50 | 41.79 | 41.77 | 41.59 | 0.11 | 0.04 |
| Replacer Intake | | | | | | | |

Period Dry Feed Intake, lb. (DM Basis)

| | | | | | | | |
|---|---|---|---|---|---|---|---|
| Week 1 | 0.19 | 0.20 | 0.25 | 0.31 | 0.34 | 0.07 | 0.29 |
| Week 2 | 1.13 | 1.18 | 1.38 | 1.90 | 1.53 | 0.36 | 0.37 |
| Week 3 | 3.37 | 3.62 | 3.87 | 4.98 | 3.94 | 0.67 | 0.34 |
| Week 4 | 6.28 | 7.51 | 7.25 | 8.96 | 7.61 | 0.89 | 0.13 |
| Total Dry Feed | 10.98 | 12.51 | 12.75 | 16.15 | 13.42 | 1.88 | 0.20 |
| Intake | | | | | | | |

Feed: Gain

| | | | | | | | |
|---|---|---|---|---|---|---|---|
| Wks. 1-4 | 1.90 | 1.82 | 1.64 | 1.72 | 7.71 | 0.09 | 0.09 |

^{a}SEM = Standard error of the mean.
^{b}Contrast P-value compare control with all other phytase levels and sources.

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates, or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

Although the present disclosure provides references to preferred embodiments, persons skilled in the art will recognize that changes may be made in form and detail without departing from the spirit and scope of the invention.

1. A method of feeding livestock animals, the method comprising:
feeding the livestock animals a milk replacer comprising non-milk proteins and phytase.

2. The method of claim 1, wherein the livestock animals ingest between about 0.01 and about 8 grams of phytase per head per day.

3. The method of claim 1, wherein the non-milk proteins comprise vegetable protein.

4. The method of claim 1, wherein the livestock animals are calves.

5. The method of claim 4, wherein the calves ingest about 0.5 to about 3.0 pounds of the milk replacer per head per day.

6. The method of claim 1, wherein the milk replacer includes about 18 to about 30 wt % protein by dry weight.

7. The method of claim 1, wherein the milk replacer includes about 15 to about 31 wt % fat by dry weight.

8. The method of claim 1, wherein the livestock animals are fed the milk replacer between about birth and about 3 weeks of age.

9. The method of claim 1, wherein the livestock animals are fed the milk replacer between about birth and about completion of the weaning process.

10. The method of claim 1, wherein the livestock animals are fed the milk replacer from about birth until weaning.

11. The method of claim 1, wherein in response to ingesting the milk replacer, the livestock animals increase a rate of weight gain.

12. The method of claim 1, wherein in response to ingesting the milk replacer, the livestock animals increase feed efficiency.

13. The method of claim 1, wherein in response to ingesting the milk replacer, the livestock animals decrease a rate of milk replacer refusal.

14. The method of claim 1, wherein the phytase is from a species of bacteria selected from a group of genera including: Escherichia, Buttiauxella, Aspergillus, or Thermomyces.

15. The method of claim 14, wherein the species of bacteria has been genetically modified.

16. The method of claim 1, further comprising activating the phytase prior to the step of feeding, wherein activating the phytase comprises combining the phytase with an aqueous material having a temperature of about 90° F. to about 160° F.

17. The method of claim 16, wherein the activated phytase rests for an activation period of about 10 to about 90 minutes prior to the step of feeding.

18. The method of claim 1, wherein the step of feeding further comprises feeding starter feed to the animals, and wherein in response to ingesting the milk replacer, the livestock animals increase consumption of the starter feed.

19. A method of feeding calves, the method comprising:
feeding the calves a milk replacer comprising non-milk proteins and phytase,
wherein the calves are between about 0 and about 3 weeks of age, and
wherein the calves increase a rate of weight gain in response to ingestion of the milk replacer.

20. The method of claim 19, wherein the calves ingest between about 0.01 and about 8 grams of phytase per head per day.
